(19) United States
(12) Patent Application Publication
(10) Pub. No.: US 2018/0125435 A1
(43) Pub. Date: May 10, 2018

(54) SIMULTANEOUS EMPLOYMENT OF DIFFERENT CONTRAST MEDIA IN CT IMAGING METHODS

(71) Applicant: Siemens Healthcare GmbH, Erlangen (DE)

(72) Inventors: Thomas FLOHR, Uehlfeld (DE); Bernhard SCHMIDT, Fuerth (DE)

(73) Assignee: Siemens Healthcare GmbH, Erlangen (DE)

(21) Appl. No.: 15/791,466

(22) Filed: Oct. 24, 2017

(30) Foreign Application Priority Data
Nov. 10, 2016 (DE) 102016222093.4

(51) Int. Cl. A61B 6/03; A61B 6/00

(52) U.S. Cl. CPC A61B 6/032; A61B 6/481; G06F 19/321; G06T 2207/10081; A61B 6/504

(57) ABSTRACT

A method is described for generating contrast medium-aided CT image data from an examination region of a patient. At least two sets of projection measurement data assigned to different X-ray energy spectra are acquired from the examination region. At least two different contrast media are present simultaneously during the acquisition in the examination region. Furthermore, at least two separate image datasets are reconstructed based upon the at least two sets of projection measurement data with the aid of a multi-material decomposition. Each of the at least two separate image datasets is assigned to one of the at least two contrast media and the materials according to which decomposition takes place are the respective contrast media. Additionally a method for analyzing morphological and/or functional parameters assigned to different contrast media in an examination region of a patient is described. Furthermore an image reconstruction device and a computed tomography system is described.

### PRIORITY STATEMENT

The present application hereby claims priority under 35 U.S.C. § 119 to German patent application number DE 102016222093.4 filed Nov. 10, 2016, the entire contents of which are hereby incorporated herein by reference.

### FIELD

At least one embodiment of the invention generally relates to a method for generating contrast medium-aided CT image data from an examination region of a patient. Moreover, at least one embodiment of the invention generally relates to a method for analyzing morphological and/or functional parameters assigned to different contrast media in an examination region of a patient. Furthermore, at least one embodiment of the invention relates to an image reconstruction device. Moreover, at least one embodiment of the invention relates to a computed tomography system.

### BACKGROUND

Modern imaging methods are frequently used to generate two-dimensional or three-dimensional image data which can be used for visualizing a mapped examination object and beyond this also for further applications.

The imaging methods are frequently based on capturing X-ray radiation, so-called projection measurement data being generated. For example projection measurement data can be acquired with the aid of a computed tomography system (CT system). In CT systems a combination of an X-ray source and an oppositely arranged X-ray detector arranged on a gantry usually runs around a measuring space in which the examination object (which is referred to below without restricting generality as the patient) is situated. The center of rotation (also referred to as the “isocenter”) coincides in this regard with a so-called system axis z. During one or more rotations the patient is exposed to X-ray radiation from the X-ray source while projection measurement data or respectively X-ray projection data is captured with the aid of the oppositely located X-ray detector, the data describing the X-ray attenuation of the patient in that direction of exposure.

The projection measurement data generated, also referred to as projection data for short, is dependent in particular on the design of the X-ray detector. X-ray detectors usually have multiple detection units which are mostly arranged in the form of a regular pixel array. The detection units generate a detection signal in each case for X-ray radiation impinging on the detection units which is analyzed at specific times in terms of intensity and spectral distribution of the X-ray radiation in order to reach conclusions about the examination object and generate projection measurement data.

In a number of types of CT imaging methods, multiple image recordings are carried out from one and the same examination region of a patient with X-ray radiation with different X-ray energy spectra. This procedure is also referred to as multi-energy CT recording. A multi-energy CT recording of this type can be affected for example with the aid of multiple CT image recordings one after another with different tube voltages. Recordings with different energy spectra can also be realized simultaneously if an energy-sensitive detector is used and X-ray attenuation data with different effective spectra is recorded simultaneously during a single CT image recording. This procedure can be realized for example with the aid of quantum-counting detectors or multi-layer detectors. These types of multi-energy CT image recordings can be utilized for example to determine the composition of body substance or respectively the proportion of different materials in an examination region.

Contrast media are frequently employed in computed tomography for improving the representation of contrasts of anatomical structures and also for identifying functional parameters from so-called 4D mappings. A contrast medium generally comprises a material which possesses a high atomic number. Examples of such a material as a constituent of a contrast medium are iodine, gadolinium, iron, and tungsten. It is often desirable to use contrast media which have different molecular diameters. For example it would be advantageous to use extracellular contrast media and intranasal contrast media in an imaging process. Extracellular contrast media have the property that they diffuse rapidly into the extravascular space due to the small size of the molecules. The permeability of tissue or respectively vessels can be determined with the aid of these types of contrast media. The porosity of vessels is changed in the event of tumors occurring for example.

Intravasal contrast media on the other hand remain in blood vessels and do not diffuse into the extravascular space or do so only very slowly. Intravasal contrast media can therefore be utilized to identify different anatomical details and tissue permeabilities than is possible with extracellular contrast media. For example the blood volume in an examination region can be determined with intranasal contrast media.

Up to now it has been customary to administer different contrast media consecutively and to record image data relating to the different contrast media at various time points, that is to say consecutively. A procedure of this type is described for example in US 2015/0 221 082 A1. However the time taken for the imaging is increased in the case of a sequential administration of the contrast media. Additionally it is not possible in the case of such a sequential imaging with the aid of different contrast media to generate the image recordings with the different contrast media in the same biological state.

Approaches also exist in which contrast media are employed simultaneously and an evaluation of the captured projection measurement data is effected with the aid of a so-called K-edge technology. However monochromatic X-ray beams and also X-ray detectors with a high level of energy resolution are necessary in this case for a precise evaluation. Neither of these preconditions is satisfied in the case of a customarily employed CT system however.

### SUMMARY

In at least one embodiment of the present invention, a method is specified for generating contrast medium-aided CT image data. Further, in at least one embodiment of the present invention, a corresponding image reconstruction device is specified with which imaging with multiple contrast media is simplified and speeded up.

At least one embodiment is directed to a method for generating contrast medium-aided CT image data from an examination region of a patient. At least one embodiment is directed to a method for analyzing morphological and/or functional parameters assigned to different contrast media in an examination region of a patient. At least one embodiment is directed to an image reconstruction device. Further, At least one embodiment is directed to a computed tomography system.

In at least one embodiment of the inventive method for generating contrast medium-aided CT image data from an examination region of a patient, at least two sets of projection measurement data assigned to different X-ray energy spectra are firstly acquired from the examination region. The statement that the two sets of projection measurement data are assigned to different X-ray energy spectra is to be understood to mean that in order to generate the different sets of projection measurement data either X-ray beams with different X-ray energy spectra were used, as is customary in the case of dual-energy or respectively multi-energy CT imaging, or different sets of projection measurement data with different spectral components were captured, which is also referred to as “spectral imaging”, with the aid of spectrum-resolving detectors, for example quantum-counting detectors. In this regard for example a different section of the X-ray energy spectrum of the X-ray beams detected during acquisition of the projection measurement data is assigned to each of the two sets.

At least one embodiment of the inventive image reconstruction device has an input interface for receiving at least two sets of projection measurement data assigned to different X-ray energy spectra from an examination region of a patient. In this regard at least two different contrast media are present in the examination region. Forming part of the inventive image reconstruction device is also an image reconstruction unit for reconstructing at least two separate image datasets on the basis of the at least two sets of projection measurement data with the aid of a multi-material decomposition, each of the at least two separate image datasets being assigned to one of the at least two contrast media and the materials according to which decomposition takes place are the respective contrast media.

At least one embodiment of the inventive computed tomography system has a scanning unit for acquiring projection measurement data from an examination region of a patient and an inventive image reconstruction device for reconstructing image data on the basis of the captured projection measurement data.

A number of fundamental components of at least one embodiment of the inventive image reconstruction device can be realized for the most part in the form of software components. This relates in particular to the image reconstruction unit. In principle however this component can also be realized partly in the form of software-supported hardware, for example FPGAs or the like, in particular where particularly fast calculations are involved. Likewise the necessary interfaces can be realized as software interfaces, for example where it is just a question of fetching data from other software components. But they can also be realized as interfaces constructed out of hardware which are activated by way of suitable software.

A largely software-based implementation has the advantage that computed tomography systems already in use up to now can also be retrofitted easily by way of a software update in order to operate in an inventive manner. To this extent, at least one embodiment is also directed to a corresponding computer program product with a computer program which can be loaded directly into a memory device of a computed tomography system, with program sections to carry out all the steps of one of embodiments of the inventive methods when the program is executed in the computed tomography system. A computer program product of this type can comprise where appropriate, alongside the computer program, additional constituents such as e.g. documentation and/or additional components, including hardware components, such as e.g. hardware keys (dongles, etc.) for utilizing the software.

Further particularly advantageous embodiments and developments of the invention arise from the dependent claims and also from the following description, it being possible for the independent claims of one claim category also to be developed analogously to the dependent claims or parts of the description of another claim category, and in particular for individual features of various example embodiments or, respectively, variants to also be combined into new example embodiments or, respectively, variants.

### BRIEF DESCRIPTION OF THE DRAWINGS

The invention is explained in detail again below while making reference to the enclosed figures on the basis of example embodiments. In this regard identical components are labeled with identical reference numbers in the various figures, in which:

FIG. 1 shows a flowchart which illustrates a method for generating contrast medium-aided CT image data according to an example embodiment of the invention,

FIG. 2 shows a schematic representation of a reconstruction device according to an example embodiment of the invention,

FIG. 3 shows a schematic representation of a computed tomography system according to an example embodiment of the invention.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. The present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “exemplary” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuity such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or porcessors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (procesor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

In at least one embodiment of the inventive method for generating contrast medium-aided CT image data from an examination region of a patient, at least two sets of projection measurement data assigned to different X-ray energy spectra are firstly acquired from the examination region. The statement that the two sets of projection measurement data are assigned to different X-ray energy spectra is to be understood to mean that in order to generate the different sets of projection measurement data either X-ray beams with different X-ray energy spectra were used, as is customary in the case of dual-energy or respectively multi-energy CT imaging, or different sets of projection measurement data with different spectral components were captured, which is also referred to as “spectral imaging”, with the aid of spectrum-resolving detectors, for example quantum-counting detectors. In this regard for example a different section of the X-ray energy spectrum of the X-ray beams detected during acquisition of the projection measurement data is assigned to each of the two sets.

During the recording of the projection measurement data at least two different contrast media are present simultaneously during the acquisition in the examination region. For example these can have been administered simultaneously to the patient in advance, i.e. prior to the start of the contrast medium-aided imaging. Different contrast media are understood in this connection to be contrast media in which the constituents of the contrast medium which are responsible for generating the contrast have a different atomic weight. Subsequently at least two separate image datasets are reconstructed on the basis of the at least two sets of projection measurement data with the aid of a multi-material decomposition.

A multi-material decomposition or basis material decomposition is described for example in PHYS. MED. BIOL., 1976, VOL. 21, NO. 5, 733-744, “Energy-selective Reconstructions in X-ray Computerized Tomography, R. E. Alvarez and A. Macovski, the entire contents of which are hereby incorporated herein by reference, for a decomposition according to two basis materials. In this regard two projection measurement datasets or image datasets are generated where the attenuation values or density values determined for the datasets correspond to the attenuation by the respective basis materials or respectively the concentration of the respective basis materials. Decomposition by basis materials can be effected both in the projection measurement data space and also in the image data space. Conventional applications of this technology use as typical basis materials iodine and water or bone and water for example, in which different scattering mechanisms, i.e. the photoelectric effect and the Compton effect, are relevant.

In place of this the two different contrast media are now used as basis materials in the case of the inventive method. In the case of a basis material decomposition of this type it is known what concentration of what basis material occurs at what location following an image data reconstruction. Therefore two separate image datasets are generated with one of the two image datasets in each case reproducing a location-resolved distribution of the concentration of one of the two contrast media.

Advantageously a basis material decomposition of this type can also be employed in the case of using polychromatic X-ray beams so that a wider field of application is created for the inventive method compared with previous methods based on the use of monochromatic X-ray radiation. Moreover image recording of different contrast media at the same time gives rise to a time saving and allows the morphological information or functional parameters which are to be determined with the aid of the different contrast media to be identified in the same biological state.

In the case of at least one embodiment of the inventive method for analyzing morphological and/or functional parameters assigned to different contrast media in an examination region of a patient the first step is to implement the inventive method for generating contrast medium-aided CT image data from an examination region of a patient. Subsequently the morphological and/or functional parameters are evaluated separately on the basis of the different image datasets, i.e. parameter values are determined for the different parameters. Morphological parameters refer to the structures and forms in the examination region which is to be mapped. Functional parameters on the other hand relate to physiological processes in the examination region. Advantageously the parameters or respectively parameter values assigned to these parameters can be determined for different simultaneously administered contrast media from separate image datasets. Therefore the database for determining the parameters or respectively parameter values is exactly as reliable as in the case of sequential image recording of images with different contrast media. Advantageously, however, time is saved and measurement in one and the same biological state becomes possible when the measurement data is captured on the basis of the simultaneous process.

At least one embodiment of the inventive image reconstruction device has an input interface for receiving at least two sets of projection measurement data assigned to different X-ray energy spectra from an examination region of a patient. In this regard at least two different contrast media are present in the examination region. Forming part of the inventive image reconstruction device is also an image reconstruction unit for reconstructing at least two separate image datasets on the basis of the at least two sets of projection measurement data with the aid of a multi-material decomposition, each of the at least two separate image datasets being assigned to one of the at least two contrast media and the materials according to which decomposition takes place are the respective contrast media.

At least one embodiment of the inventive computed tomography system has a scanning unit for acquiring projection measurement data from an examination region of a patient and an inventive image reconstruction device for reconstructing image data on the basis of the captured projection measurement data.

A number of fundamental components of at least one embodiment of the inventive image reconstruction device can be realized for the most part in the form of software components. This relates in particular to the image reconstruction unit. In principle however this component can also be realized partly in the form of software-supported hardware, for example FPGAs or the like, in particular where particularly fast calculations are involved. Likewise the necessary interfaces can be realized as software interfaces, for example where it is just a question of fetching data from other software components. But they can also be realized as interfaces constructed out of hardware which are activated by way of suitable software.

A largely software-based implementation has the advantage that computed tomography systems already in use up to now can also be retrofitted easily by way of a software update in order to operate in an inventive manner. To this extent, at least one embodiment is also directed to a corresponding computer program product with a computer program which can be loaded directly into a memory device of a computed tomography system, with program sections to carry out all the steps of one of embodiments of the inventive methods when the program is executed in the computed tomography system. A computer program product of this type can comprise where appropriate, alongside the computer program, additional constituents such as e.g. documentation and/or additional components, including hardware components, such as e.g. hardware keys (dongles, etc.) for utilizing the software.

For transport to the computed tomography system and/or for storage on or in the computed tomography system, use can be made of a computer-readable medium, for example a memory stick, a hard drive, or some other transportable or permanently installed data carrier on which are stored the program sections of the computer program which can be read in and executed by an arithmetic-logic unit of the computed tomography system. To this end the arithmetic-logic unit can have for example one or more interoperating microprocessors or the like.

In one embodiment of the inventive method for generating contrast medium-aided CT image data from an examination region of a patient, the at least two different contrast media have different molecular sizes. Contrast media with different molecular sizes can be utilized for determining different anatomical details and tissue permeabilities simultaneously. Advantageously this operation can be effected in a shorter time than in the case of a sequential procedure.

In a particularly practical embodiment of the inventive method for generating contrast medium-aided CT image data from an examination region of a patient the same time point or time points lying shortly after each other are chosen for the individual contrast media for the purpose of an injection of the contrast media used during the acquisition of the projection measurement data. Advantageously a simultaneous appearance of the different contrast media in the examination region can be achieved with such a procedure so that the image recording of the examination region can be effected simultaneously for the two contrast media.

In an embodiment of the inventive method for generating contrast medium-aided CT image data from an examination region of a patient a two-material decomposition is used as the multi-material decomposition. A two-material decomposition makes sense in the application of exactly two contrast media since functional or morphological parameters or respectively parameter values assigned to two different contrast media can therefore be determined simultaneously.

In an alternative embodiment of the inventive method for generating contrast medium-aided CT image data from an examination region of a patient, a three-material decomposition is used as the multi-material decomposition. A three-material decomposition makes sense in the application of exactly three contrast media since functional or morphological parameters or respectively parameter values assigned to three different contrast media can therefore be determined simultaneously.

In an embodiment of the inventive method for generating contrast medium-aided CT image data from an examination region of a patient, the acquisition of the projection measurement data is effected in the context of a static image recording. Static image recordings can be used for example for identifying blood volume images. A blood volume image can be employed for example for determining tissue anomalies and sample excision sites.

In an alternative variant of the inventive method for generating contrast medium-aided CT image data from an examination region of a patient, the acquisition of the projection measurement data is effected in the context of a dynamic image recording. In a dynamic image recording more complex functional variables can be determined, such as for example permeability or flow.

In a special embodiment of the inventive method for generating contrast medium-aided CT image data from an examination region of a patient, a first contrast medium of the at least two contrast media comprises an extracellular contrast medium and a second of the at least two contrast media an intranasal contrast medium. Extracellular contrast media usually have small molecular sizes and consequently diffuse rapidly into the extravascular space. The porosity of vessels for example can be determined with this type of contrast medium. Intravasal contrast media have larger molecular dimensions and consequently remain in the blood vessels and do not diffuse into the extravascular space or do so only very slowly.

Structures can be mapped in the stationary state with this kind of contrast medium. Since they circulate in the blood vessels for longer they are suitable for mapping both arteries and also veins. Further fields of application for intravasal contrast media are: detecting intestinal hemorrhaging, visualizing blood vessels of tumors, measuring blood volume, measuring perfusion, and detecting endovascular leaks.

Therefore morphological and functional parameters which can only be captured with different contrast media with different molecular sizes can be determined simultaneously in this advantageous embodiment.

In a particularly practical embodiment of the inventive method for generating contrast medium-aided CT image data from an examination region of a patient, at least two contrast media are used which in each case comprise at least one of the following materials:
iodine,
gadolinium,
iron
tungsten.

The different materials have different atomic weights and consequently also give rise to different spectral absorption behavior. The atom types can in each case form part of extracellular contrast media or intravasal contrast media. The diffusion behavior of the contrast media on the other hand is defined, as already mentioned, by the size of the contrast medium molecules, which is only influenced to a minor degree by the type of the materials responsible for the contrast effect.

In a preferred embodiment of the inventive method for analyzing morphological and/or functional parameters assigned to different contrast media in an examination region of a patient, the morphological and/or functional parameters comprise at least one of the following parameters:
blood volume,
permeability,
blood flow.

Whereas blood volume is determined by static image recordings, functional parameters such as permeability and blood flow are determined with the aid of dynamic image recordings in which an examination region is captured in imaging over the course of a predetermined time period.

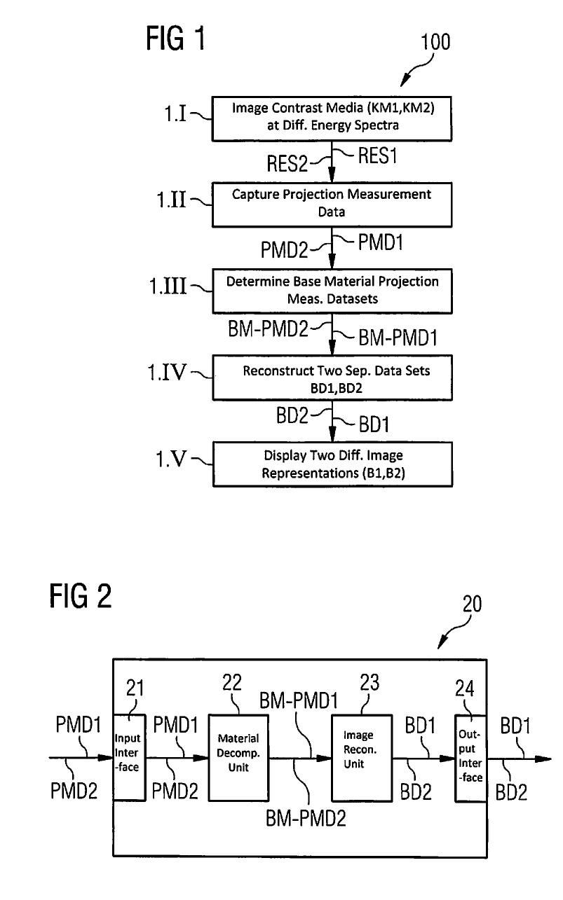

FIG. 1 shows a flowchart 100 which illustrates a method for generating contrast medium-aided CT image data according to an example embodiment of the invention. In advance, i.e. prior to the start of the method two different contrast media KM1, KM2 with different molecular sizes and therefore different diffusion behaviors were injected at approximately the same time in a patient to be examined. In the example embodiment illustrated in FIG. 1 the first of the two different contrast media KM1 comprises iodine and the second of the two different contrast media KM2 gadolinium. Once the two contrast media have both reached an examination region at approximately the same time a CT imaging method with two different X-ray energy spectra RES1, RES2 is started in step 1.I. Furthermore in step 1.II projection measurement data PMD1, PMD2 with the two different X-ray energy spectra RES1, RES2 are captured from the examination region while the two different contrast media KM1, KM2 are present simultaneously in the examination region. In step 1.III a determination of basis material projection measurement datasets BM-PMD1, BM-PMD2 is effected on the basis of the two projection measurement datasets PMD1, PMD2. Advantageously in this regard the two different contrast media KM1, KM2 are used as basis materials in the example embodiment shown in FIG. 1. For this purpose line integrals A1, A2 of the absorption and therefore of the densities ρ1(x, y, z), ρ2(x, y, z) of the contrast media KM1, KM2 are determined for each projection direction on the basis of the two acquired projection measurement datasets PMD1, PMD2. Lastly in step 1.IV two separate image datasets BD1, BD2 are reconstructed with the aid of a filtered back projection on the basis of the two basis material projection measurement datasets BM-PMD1, BM-PMD2, the image datasets being assigned to one of the two contrast media KM1, KM2 in each case. In step 1.V two different image representations B1, B2, which are based on one of the two image datasets BD1, BD2 in each case, are displayed to a user.

FIG. 2 illustrates a schematic representation of a reconstruction device 20 according to an example embodiment of the invention. The reconstruction device 20 comprises an input interface 21. The input interface 21 receives projection measurement data PMD1, PMD2, which is assigned to different X-ray energy spectra, from an examination region of a patient and passes the projection measurement data PMD1, PMD2 on to a material decomposition unit 22, which generates basis material projection measurement datasets BM-PMD1, BM-PMD2 in the previously described manner on the basis of the received projection measurement data PMD1, PMD2. The basis material projection measurement datasets BM-PMD1, BM-PMD2 are transferred to an image reconstruction unit 23 which reconstructs two separate image datasets BD1, BD2 on the basis of the basis material projection measurement datasets BM-PMD1, BM-PMD2. The image datasets BD1, BD2 which are determined are transferred to other units via an output interface 24, such as for example a data storage unit or an image display unit.

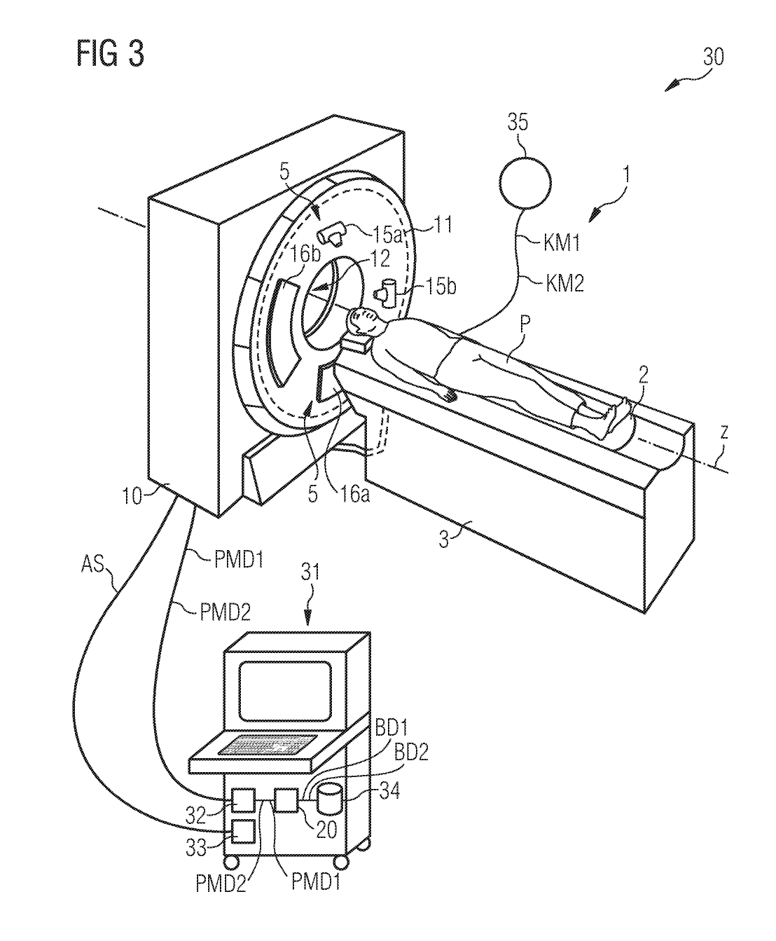

FIG. 3 shows a computed tomography system 30 which comprises the reconstruction device 20 shown in FIG. 2. In this regard the CT system 1 essentially consists of a normal scanning unit 10 in which, on a gantry 11, a projection data acquisition unit 5 with two detectors 16a, 16b and two X-ray sources 15a, 15b which are respectively located opposite the two detectors 16a, 16b rotates around a measuring space 12. Situated in front of the scanning unit 10 is a patient support device 3 or respectively a patient table 3, the upper part 2 of which can be traversed, with a patient P situated on it, to the scanning unit 10 in order to move the patient P through the measuring space 12 relative to the detectors 16a, 16b. The scanning unit 10 and the patient table 3 are activated by a control device 31 from which acquisition control signals AS come via a normal control interface 33 in order to activate the entire system according to predetermined measuring protocols in the conventional manner. In the case of a spiral acquisition a movement of the patient P along the z direction, which corresponds to the system axis z longitudinally through the measuring space 12, and the simultaneous rotation of the X-ray sources 15a, 15b give rise to a helical path for the X-ray sources 15a, 15b relative to the patient P during the measurement. In parallel the oppositely located detector 16a, 16b always runs in step opposite the X-ray sources 15a, 15b in this regard in order to capture projection measurement data PMD1, PMD2, which is then utilized for reconstructing dual-energy volume and/or layer image data. Likewise a sequential measuring method can also be carried out in which a fixed position in the z direction is traveled to and then the required projection measurement data PMD1, PMD2 is captured at the relevant z position during a rotation, a partial rotation, or multiple rotations in order to reconstruct a sectional image at this z position, or to reconstruct image data from the projection data for multiple z positions. The inventive method is fundamentally also capable of being employed on other CT systems, e.g. with a detector forming a complete ring. For example the inventive method can also be applied on a system with a non-moving patient table and a gantry moving in the z direction (a so-called sliding gantry).

Additionally FIG. 3 shows a contrast medium injection unit 35 which is set up to inject the patient P with at least two different contrast media KM1, KM2 in advance, i.e. prior to the start of a CT imaging method.

The projection measurement data PMD1, PMD2 (also referred to as raw data) acquired by the two detectors 16a, 16b is handed on via a raw data interface 32 to the control device 31. This projection measurement data then undergoes further processing, where appropriate after suitable pre-processing (e.g. filtering and/or beam hardening correction), in an inventive image reconstruction device 20 which, in this example embodiment, is implemented in the control device 31 in the form of software on a processor. With the aid of the reconstruction method described in connection with FIG. 1 this image reconstruction device 20 reconstructs image data BD1, BD2 on the basis of the projection measurement data PMD1, PMD2.

The reconstructed image data BD1, BD2 is then transferred to an image data storage unit 34, from which it is transferred for example to an image display unit for pictorial representation. Via an interface not shown in FIG. 3 it can also be fed into a network connected to the computed tomography system 1, for example a radiological information system (RIS), and deposited in a mass memory which is accessible there, or output as images on printers or filming stations which are connected there. Thus the data can be subjected to further processing in any desired fashion and then stored or output.

The components of the image reconstruction unit 20 can be implemented for the most part or entirely in the form of software elements on a suitable processor. In particular the interfaces between these components can also be realized purely out of software. What is required is just the existence of options for accessing suitable storage regions in which the data is suitably held in buffer storage and can be called up again and updated at any time.

Finally attention is drawn once again to the fact that the medical technology apparatuses and methods described in detail in the foregoing just concern example embodiments which can be modified in the most varied ways by a person skilled in the art without departing from the scope of the invention.

Furthermore the use of the indefinite article “a” or respectively “an” does not exclude the eventuality that the relevant features can also be present in multiple instances. Nor is it excluded that elements of the present invention represented as individual units consist of multiple interacting subcomponents which can also be spatially distributed where appropriate.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. § 112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for generating contrast medium-aided CT image data from an examination region of a patient, the method comprising:
acquiring at least two sets of projection measurement data respectively assigned to different X-ray energy spectra from the examination region, wherein at least two different contrast media are present simultaneously in the examination region during the acquiring; and
reconstructing at least two separate image datasets based upon the at least two sets of projection measurement data, with the aid of a multi-material decomposition, wherein each of the at least two separate image datasets is assigned to a respective one of the at least two different contrast media and materials, according to which the multi-material decomposition is to take place, are the respective at least two different contrast media.

2. The method of claim 1, wherein the at least two different contrast media have different molecular sizes.

3. The method of claim 1, wherein a same time point, or time points lying shortly after each other, are chosen for individual contrast media of the at least two different contrast media for a purpose of an injection of the respective individual contrast media present during the acquiring of the at least two sets of projection measurement data.

4. The method of claim 1, wherein a two-material decomposition is used as the multi-material decomposition.

5. The method of claim 1, wherein a three-material decomposition is used as the multi-material decomposition.

6. The method of claim 1, wherein the acquiring of the at least two sets of projection measurement data is affected in the context of a static image recording.

7. The method of claim 1, wherein the acquiring of the at least two sets of projection measurement data is affected in the context of a dynamic image recording.

8. The method of claim 1, wherein a first contrast medium of the at least two different contrast media comprises an extracellular contrast medium and a second contrast medium of the at least two different contrast media comprises an intranasal contrast medium.

9. The method of claim 1, wherein the at least two different contrast media include at least one of the following materials:
iodine,
gadolinium, and
tungsten.

10. A method for analyzing at least one of morphological and functional parameters assigned to different contrast media in an examination region of a patient, the method comprising:
implementing the method of claim 1; and
separating an evaluation of at least one of morphological and functional parameters in the at least two separate image datasets.

11. The method of claim 10, wherein the at least one of morphological and functional parameters comprise at least one of the following parameters:
blood volume,
permeability, and
blood flow.

12. An image reconstruction device, comprising:
an input interface to receive at least two sets of projection measurement data assigned to different X-ray energy spectra from an examination region of a patient, wherein at least two contrast media with different molecular sizes are present in the examination region; and
an image reconstruction unit to reconstruct at least two separate image datasets based upon the at least two sets of projection measurement data with the aid of a multi-material decomposition, wherein each of the at least two separate image datasets is assigned to a respective one of the at least two contrast media and wherein materials, according to which the multi-material decomposition is to take place, are the respective at least two contrast media.

13. A computed tomography system, comprising:
a scanning unit to acquire the at least two sets of projection measurement data from an examination region of a patient; and
the image reconstruction device of claim 12.

14. A non-transitory computer program product including a computer program, directly loadable into a memory device of a computed tomography system, the computer program including program sections to carry out the method of claim 1 in response to the computer program being executed in the computed tomography system.

15. A non-transitory computer-readable medium, including program sections stored thereon which are readable and executable by an arithmetic-logic unit to carry out the method of claim 1 in response to the program sections being executed by the arithmetic-logic unit.

16. The method of claim 2, wherein a same time point, or time points lying shortly after each other, are chosen for individual contrast media of the at least two different contrast media for a purpose of an injection of the respective individual contrast media present during the acquiring of the at least two sets of projection measurement data.

17. The method of claim 2, wherein a two-material decomposition is used as the multi-material decomposition.

18. The method of claim 3, wherein a three-material decomposition is used as the multi-material decomposition.

19. The method of claim 2, wherein a first contrast medium of the at least two different contrast media comprises an extracellular contrast medium and a second contrast medium of the at least two different contrast media comprises an intranasal contrast medium.
